(19) United States
(12) Patent Application Publication
(10) Pub. No.: US 2013/0310355 A1
(43) Pub. Date: Nov. 21, 2013

(54) LOW TOXICITY TOPICAL ACTIVE AGENT DELIVERY SYSTEM

(71) Applicant: YOUNG PHARMACEUTICALS, INC., Wethersfield, CT (US)

(72) Inventor: John E. Kulesza, Berlin, CT (US)

(21) Appl. No.: 13/795,663

(22) Filed: Mar. 12, 2013

Related U.S. Application Data
(63) Continuation-in-part of application No. 13/685,244, filed on Nov. 26, 2012.
Application No. 13/685,244 is a continuation of application No. 12/711,381, filed on Feb. 24, 2010, now Pat. No. 8,337,870.
(60) Provisional application No. 61/717,830, filed on Oct. 24, 2012.
(60) Provisional application No. 61/286,668, filed on Dec. 15, 2009.

(51) Int. Cl. A61K 47/34; A61K 31/57; A61K 31/215

(52) U.S. Cl. CPC A61K 47/34; A61K 31/215; A61K 31/57
USPC 514/180; 514/529

(57) ABSTRACT

An active agent delivery composition is provided that allows topical delivery of active agents including vitamin A and its derivatives. A volatile vehicle serves as a coupler for an active agent and an organosiloxane carrier so as to allow full solubilization of active agents not normally miscible in silicones and providing a non-irritating, targeted evaporating composition.

### CROSS REFERENCE TO RELATED APPLICATIONS

This application depends from and claims priority to U.S. Provisional Application No. 61/717,830 filed Oct. 24, 2012, and this application is a continuation-in-part of U.S. application. Ser. No. 13/685,244 filed Nov. 26, 2012, which is a continuation of U.S. application Ser. No. 12/711,381 filed Feb. 24, 2010, which claims priority to U.S. Provisional Application No. 61/286,668 filed Dec. 15, 2009, the entire contents of each of which are incorporated herein by reference.

### FIELD OF THE INVENTION

The invention relates to compositions for topical delivery of active agents. The compositions relate to delivery of active agents to the skin with reduced toxicity such as drying, irritation, or inflammation. The inventive composition is related to delivery of topical agents such as retinoids to the skin.

### BACKGROUND OF THE INVENTION

The comfort associated with application of topical agents is related in part to the rate of evaporation of the applied composition on the skin. A product with a slow evaporation rate could feel greasy on the skin whereas a product with an overly rapid evaporation rate feels either as if it has not been applied to the skin at all or leaves the user with the impression that not enough has been applied possibly leading to overuse. Combining topical agents with volatile silicones has been found to provide a pleasing application. Silicones and other organosiloxanes, however, by themselves are poor solvents for hydrophobic organic active agents.

To address the poor solubility in silicones, delivery systems for these agents commonly require 35% or more organic solvent as a carrier to solubilize the active agent as well as provide suitability for combination with volatile compounds that provide pleasing application by the user. Prior formulations prefer alcohols such as ethyl alcohol as an organic solvent to solubilize the active agent in a silicone.

Lipophilic skin care active agents such as retinoids are generally applied topically to reduce the appearance of aging, for other cosmetic purposes, or to treat a skin condition such as acne. The retinoids, however, along with many other active agents contribute to thinning or drying of the skin. This problem is worsened by including significant levels of organic solvents that themselves can alter epidermal barrier lipids and contribute to skin irritation. Ethyl alcohol, as used in the retinoid composition of U.S. Pat. No. 4,826,828 was believed to be the solution for topical delivery of hydrophobic agents. Ethyl alcohol, to the contrary, contributes to skin irritation and dryness. Thus, combining ethyl alcohol with potentially irritating active agents increases skin dryness leading to non-optimal use.

Thus, there exists a need for a hydrophobic active agent delivery system that provides pleasing application and does not contribute to toxicity.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

An active agent delivery composition is provided that creates pleasing administration of an active agent to the skin of a subject without a greasy feeling residue, includes only non-irritating solution components at their concentrations, and is a clear solution alone, an optionally in the presence of one or more additives. The achievement of these three characteristics of an active agent delivery composition is provided by the function of a non-irritating volatile vehicle that will promote solubilization of the active agent in an organosiloxanes carrier as a primary solvent. In many embodiments, the ability of the volatile vehicle to produce a clear, fully solubilized solution is surprising in that in many embodiments the active agent is not fully soluble in either the organosiloxane carrier or the vehicle alone at the concentrations used in the composition.

An inventive composition includes an active agent illustratively: vitamin A or its derivatives; hydroxy acids; benzoyl peroxide; resorcinol; antimicrobials; anti-neoplastic agents; anti-viral agents; steroidal or nonsteroidal anti-inflammatory agents; UV filters; lipids; and immunomodulators. An active agent is optionally vitamin A or a derivative thereof present at between 0.001 to 2 weight percent. Optionally, a vitamin A derivative is a retinoid. A retinoid is optionally retinal, retinoic acid, retinyl ester, retinol, tretinoin or esters thereof, isotretinoin or esters thereof, adapalene, tazarotene, or combinations thereof. An active agent is optionally salicylic acid, acetylsalicylic acid, or combinations thereof.

The inventive composition provides pleasing skin administration through the use of an organosiloxane carrier that is optionally a linear aliphatic polyorganosiloxane, optionally ethyl trisiloxane, combined with an volatile vehicle with a complimentary volatility relative to the carrier such that the overall composition may have an evaporation rate in air of 10 to 500 mg/cm²/minute at 25° C. and atmospheric pressure of 760 mmHg.

A volatile vehicle is present in the inventive composition to solubilize the active agent with the carrier while reducing or eliminating the need for an organic hydrocarbon solvent such as ethanol. A vehicle is optionally a linear, branched, and/or ring formed perfluoro C₁-C₂₀ alkyl. In some embodiments, a vehicle is a perfluoro C₄-C₈ alkyl, optionally a perfluoro C₆ alkyl such as perfluorohexane. A vehicle is optionally a perfluoro ether, optionally, methoxynonafluorobutane or ethoxynonafluorobutane. A vehicle is optionally perfluorohexane, perfluorodecalin, methoxynonafluorobutane, pentafluoropropane, disiloxane, isododecane, isododecane in combination with an organic hydrocarbon solvent of 6 carbons or fewer and at a concentration of less than 15% per weight, a linear or branched C₁₂-C₁₆ alkane other than isododecane, or any combination thereof. The vehicle is optionally present from about 15 to 25 percent by weight.

An optional organic hydrocarbon solvent of 6 carbon atoms or fewer is optionally included or is absent. Optionally, the organic hydrocarbon solvent is present at less than 15 percent by weight. Optionally, the organic hydrocarbon solvent is present at less than 5 percent by weight. The composition satisfies the long felt need for a system of active agent delivery that does not require an alcohol solvent at concentrations in excess of 15%, which in the prior understanding was more typically of 30% or greater, to solubilize the active agent in an organosiloxane carrier. Thus, the resulting compositions do not suffer the skin irritancy produced by such concentrations of organic hydrocarbon solvent, and helps reduce any irritancy that is may be a characteristic of the active agent itself.

Also provided are processes of treating a skin condition in a subject. The skin condition may be, for example, acne, wrinkles, dryness, cancer, or perspiration. The inventive process includes applying an inventive composition to the skin of a subject

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art. It is to be understood that the present invention is not limited to particular embodiments described, which may, of course, vary.

The invention has utility for topical delivery of active agents. The invention has more specific utility for the delivery of hydrophobic active agents to the skin with reduced agent or solvent related side effects and improved comfort and user compliance. Some embodiments have utility for the treatment of one or more conditions of the skin. Also provided are embodiments that have utility for fully solubilizing a hydrophobic active agent in a silicone providing a clear solution with 15% or less of organic hydrocarbon having 6 carbons or fewer.

The inventive composition includes an active agent combined with an organosiloxane carrier and a compatible volatile vehicle.

As used herein the term “active agent” refers to a molecule suitable for delivery to the skin or mucosal regions of a subject. Optionally, an active agent has pharmaceutical activity and is present for the treatment or prevention of a skin condition. Active agents are optionally low polarity molecules such as those having a hydrocarbon chain of three or more carbons, but may also include materials of higher polarity. Examples of active agents illustratively include: vitamin A or its derivatives; hydroxy acids; aromatic molecules such as benzoyl peroxide and resorcinol; azelaic acid; antimicrobials such as azelaic acid, erythromycin, sodium sulfacetamide, tetracycline and derivatives, and clindamycin; anti-neoplastic agents and/or ophthalmic agents illustratively including 5-fluorouracil, doxorubicin, imiquimod, and sodium[o-(2,6-dichloranilino)phenyl]acetate; anti-viral agents illustratively ganciclovir, trifluorothymidine and related compounds; steroidal and nonsteroidal anti-inflammatory agents illustratively flurbiprofen, ibuprofen, naproxen, indomethacin and related compounds; anti-mitotic drugs illustratively colchicine taxol and related compounds; drugs that act on actin polymerization illustratively phalloidin, cytochlasin B and related compounds; inhibitors of dihydropyrimidine dehydrogenase (DPD), thymidine phosphorylase (TP) and/or uridine phosphorylase (UP) enzyme inhibitors; ultraviolet light (UV) filters illustratively benzophenone derivatives such as oxybenzone, octocrylene, octyl methoxycinnamate, and avobenzone; radiation proactive agents illustratively methyluracils such as 6-methyluracil and 4-methyluracil; and immunomodulating molecules such as tacrolimus, and pimecrolimus. An active agent need not have pharmaceutical activity. Other active agents are illustratively cosmetics such as pigments, dyes, and fillers. It is appreciated that an inventive composition optionally includes more than one active agent. Optionally, 2, 3, 4, 5, 6, or more active agents are present in an inventive composition. An active agent is optionally a prodrug that is converted to a desired active species optionally in the skin or layer thereof.

An active agent is optionally a lipid such as those suitable for controlling perspiration. Lipids optionally have an HLB of less than about 12, less than about 8, or optionally less than about 6. Illustrative examples of lipids include glyceryl monostearate, glyceryl monoisostearate, glyceryl monomyristate, glyceryl monoleate, diglyceryl monoisostearate, propylene of glycol monostearate, propylene glycol monoisostearate, propylene glycol monocaprylate, sorbitan monoisostearate, sorbitan monocaprylate, sorbitan monoisooleate, glyceryl monolaurate, glyceryl monocaprylate, glyceryl monocaprate, mixtures thereof or the like. Optionally, the lipid is glyceryl monolaurate, made available by suppliers like Fitz Chem Corporation under the name MONOMULS 90-L12.

Typically, the lipid, when present, makes up from about 4% to about 35%, and optionally, from about 5% to about 20%, and optionally, from about 10% to about 15% by weight of the composition, based on total weight of the composition and including all ranges subsumed therein.

An active agent is optionally vitamin A or a derivative of vitamin A. Examples of vitamin A or its derivatives illustratively include retinoids such as retinal, retinoic acid, retinyl ester, retinol, tretinoin or esters or amides thereof; isotretinoin or esters or amides thereof; adapalene, tazarotene, C9- or C13-cis retinoids or their derivatives (e.g. esters or amides), C13 trans retinoids or their derivatives (e.g. esters or amides), and the like. Specific examples of retinoids include those described in U.S. Pat. Nos. 5,837,728, 4,677,120 and 4,885,311. One specific example of a derivative of vitamin A is hydroxypinacolone retinoate.

Examples of hydroxy acids illustratively include beta hydroxy acids such as salicylic acid, acetylsalicylic acid, and the like may be substituted for or combined with the retinoid.

While the description uses retinoids as illustrative examples of an active agent, the specification is not limited as such. Other active agents are similarly operable herein. It is appreciated that other active agents are similarly substitutable with the retinoid, for example benzyol peroxide, or other active agent.

The compositions of the invention may include 0.005 to 1.0 weight percent retinoid, in which case they are optionally applied directly to the skin, or supplied as a more concentrated solution containing higher levels of active agent, in which case prior to application they are diluted optionally by means of a cosmetically acceptable carrier to a desired level such as 0.005 to 1.0 weight percent for retinoid. In the formulations of the invention, water is optionally minimized or eliminated to improve the stability of retinoid and to minimize the potential for separation of the oil and water. Optionally, water is present at less than 2%. One of ordinary skill in the art will recognize that differing levels of active agent will be operable herein depending on the desired final amount of active agent to be administered.

Optionally, active agent is present in less than 30 percent w/w amounts. Optionally, active agent is present at a weight percent of 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.5, 0.1, 0.01, 0.001, 0.0001, or any level or range therebetween. Optionally, active agent is present at 20 percent w/w. Illustratively, when azelaic acid is an active agent it is present at 15 to 25 percent w/w. A vitamin A derivative is optionally present at 0.001 to 2 percent by weight. Imiquimod is optionally present at 3 to 8 percent by weight. Benzoyl peroxide is optionally present at 1 to 10 percent by weight. It is within the skill of the art to determine the optimal level of active agent in either a concentrated solution or a final solution for application.

An active agent is provided in a carrier. A carrier is optionally present from 10 to 75 percent w/v. In typical embodiments, a carrier is present as a predominant component in a composition. Optionally, a carrier is present at 50% or greater w/v. Carriers are optionally volatile compounds such as volatile silicones. The use of a volatile carrier allows application of the active agent to the skin and is capable of evaporating so as to leave a pleasing feel to the user. Among the many possible carriers, the siloxanes offer excellent evaporation properties and are preferred. Siloxanes are illustratively cyclic organosiloxanes or non-cyclic organosiloxanes. Examples of cyclic organosiloxanes illustratively include cyclic polydiorganosiloxanes, cyclotetradimethicones and cyclopentadimethicones. Linear organopolysiloxanes are illustratively alkyl-, alkoxy- or phenyldimethicones, and alkyl-, alkoxy- or phenyltrimethicones. Optionally, a carrier is an aliphatic volatile organosiloxane. Aliphatic volatile organosiloxanes optionally have from two to six silicon atoms. Optionally, an aliphatic volatile organosiloxane is a linear polyorganosiloxane such as a polyorganosiloxane with 2 to 6 silicon atoms, optionally, an organo-trisiloxane, disiloxane, or combinations thereof. Disiloxane itself has a very rapid evaporation profile on the skin. As such, it is optionally not used a as a carrier. Optionally, a carrier is ethyl trisiloxane. It is appreciated that an inventive composition optionally includes more than one carrier.

Suitable organosiloxane carriers preferably are a fluid that can evaporate on contact with the skin in less than one hour in a surrounding air environment at room temperature (25° C.) and standard atmospheric pressure (760 mmHg). An organosiloxane carrier is liquid at room temperature. An organosiloxane carrier optionally has an evaporation rate ranging from 10 to 500 mg/cm²/minute, at 25° C. and atmospheric pressure of 760 mmHg. In some embodiments, the organosiloxane carriers used in the invention have an evaporation rate ranging from 20 to 200 mg/cm²/minute at 25° C. and atmospheric pressure of 760 mmHg. In some embodiments, the organosiloxane carriers used in the invention have an evaporation rate ranging from 100 to 200 mg/sq cm/minute at 25° C. and atmospheric pressure of 760 mmHg.

Volatile organosiloxanes optionally are lightweight carriers that evaporate on application and thus have an elegant, light-weight “feel” on the skin. Volatile organosiloxanes, however, are typically limited in their ability to dissolve low polarity (i.e. usually greater than C7-C8)) organic compounds like retinoids. For example, when relatively low therapeutic levels of retinol (0.1-0.2% w/v) are dissolved in cyclomethicone alone, hazy solutions result due to incomplete solubilization in the organosiloxane. To address this issue vehicles were sought that would be both capable of creating clear solutions of active agent in a volatile organosiloxane carrier, but also have an appropriate evaporation rate in combination with the volatile organosiloxane when placed on the skin so as maintain the light-weight feel on the skin experienced by a user. A vehicle must also not promote unacceptable skin irritation when used at the concentration necessary in the composition.

Traditional methods of solubilizing active agents in a volatile organosiloxane used volatile low molecular weight hydrocarbons as an organic solvent, such as ethanol. This required high hydrocarbon concentrations typically of 30% or greater. While these organic solvents did promote solubilization of active agents in the silicone carriers, they also promote undue skin dryness and irritation. When combined with a potentially irritating active agent such as benzyol peroxide, retinoids, or others, this results in a negative experience for the user. Thus, a vehicle must also not promote skin irritation when used at the final concentration in the material.

Among the nearly infinite possibilities of vehicles to select from when searching for a material that could function with both an active agent and a volatile organosiloxane, it was unexpectedly discovered that an organic polyhalogenic vehicle alone or in combination with less than 5% low molecular weight hydrocarbon organic solvent (6 carbons or fewer), or particular volatile hydrocarbons in combination with less than 5% low molecular weight hydrocarbon organic solvent (6 carbons or fewer) could incorporate an active agent such as a retinoid at appropriate therapeutic levels and reduce the levels of low molecular weight hydrocarbon solvent to less than 5 percent in contrast to U.S. Pat. No. 4,826,828, which required 35-60 percent w/w hydrocarbon solvent. It was particularly surprising that such a combination will work to solubilize an active agent as the organic polyhalogenic materials, the volatile hydrocarbons, and the carriers are unable to fully solubilize the active agent at the therapeutic concentration alone, particularly at low temperature such as 4° C. at which the active agent will crystallize and fall out of solution in either of the carrier or other vehicle materials alone. Thus, there is no expectation that the combination of the two material types, each of which do not allow full, stable, solub lization of the active agent, will do so when the materials are combined.

The discovery of using organic polyhalogenic vehicles to promote solubility in organosiloxane carriers is unexpected due to the fact that organic polyhalogenic vehicles are poor solubilizers of molecules such as retinoid on their own. Combined with the knowledge that organosiloxanes are poor solvents, one of ordinary skill in the art has no expectation of success combining two poor solubilizers to form a system that effectively and fully solubilizes active agents. Organic polyhalogenic solvents are optionally those disclosed in U.S. Pat. No. 6,251,375. In particular instances, vehicles incorporate a halogen such as one or more fluorine atoms. A vehicle is optionally a substituent containing C₁-C₂₀ alkyl, alkenyl, or alkynl, any of which is cyclic, bicyclic, linear, branched, or combinations thereof, wherein a substitutent is a fluorine atom. A vehicle is optionally a fluorine substituent containing molecule that is an aryl, a C₁-C₂₀ alkyl, a C₂-C₂₀ alkenyl, or a C₂-C₂₀ alkynyl, wherein the molecule optionally includes a second substituent that is N, O, or S. In some embodiments, a vehicle is a perfluoro C₄-C₁₂ alkyl, optionally a perfluoro C₆ alkyl such as perfluorohexane available from F2 Chemicals Ltd. (Lancashire, UK). In some embodiments, a vehicle is a bicyclic perfluoro molecule such as perfluorodecalin available from F2 Chemicals Ltd. In some embodiments, the use of perfluorodecalin produces a final composition with too low an evaporation rate resulting in a greasy feeling on the skin. As such, perfluorodecalin is optionally excluded. In some specific instances, a vehicle is a perfluoro ether. In some particular instances, a vehicle is methoxynonafluorobutane or ethoxynonafluorobutane available from 3M Specialty Materials, St. Paul, Minn. Other illustrative examples of pefluoro vehicles include: perfluorodimethylcyclohexane available from F2 Chemicals Ltd.; perfluoromethyldecalin available from F2 Chemicals Ltd.; perfluoropentane available from Fluoromed, Round Rock, Tex.); or perfluoroperhydrophenanthrene available from F2 Chemicals Ltd. Optionally, a combination of one or more polyhalogenic compounds are used to form a vehicle. Illustratively, a vehicle may be a blend of two or more of perfluorohexane, perfluorodecalin, pentafluoropropane, perfluorodimethylcyclohexane, or perfluoroperhydrophenanthrene. Optionally, a vehicle is a blend of perfluorohexane, perfluorodecalin, and pentafluoropropane, sold as Fiflow BB61 sold by the Innovation Company, France.

In some embodiments, a vehicle includes a particular volatile silicon containing material alone or in combination with a volatile low molecular weight hydrocarbon. Illustrative volatile silicon containing materials useful as a vehicle include disiloxanes (optionally disiloxane at 0.65-5 cSt), among others. In some embodiments, a vehicle is not ethyl trisiloxane. Optionally, a vehicle is disiloxane at 5-15% by weight in combination with a volatile low molecular weight hydrocarbon present at 5% or less by weight such as ethanol. It was discovered that the presence of disiloxane will promote the appropriate evaporation profile and can reduce the amount of organic solvent (e.g. ethanol) to less than 5% by weight necessary to produce fully solubilized active agent, thus promoting the pleasing feel on the skin and creating complete solubilization (i.e. clear solution) of the active agent in the carrier. This represents a delicate balance of materials such that the volatile components (e.g. 0.25-1 ml) will evaporate from the skin preferably in 20-60 seconds, optionally 20-30 seconds while simultaneously keeping the level of organic solvent at a level that will not irritate the skin.

In some embodiments, a vehicle includes a volatile high molecular weight hydrocarbon such as linear or branched volatile hydrocarbons with 6 to 16 carbon atoms, alone, combined with a second or additional vehicle with a lower boiling point, or combined with less than 5% of organic solvent of 5 carbons or fewer. The usefulness of this vehicle type was also unexpected due to the inability of such a vehicle to solubilize the active agent at low temperatures such as 4° C. for a sufficient period of time (i.e. 30 days). A volatile high molecular weight hydrocarbon vehicle is linear or branched and illustratively includes C₈-C₁₆ alkanes, branched C₈-C₁₆ esters, and mixtures thereof. Optionally, a volatile high molecular weight hydrocarbon vehicle is linear or branched and illustratively includes C₁₀-C₁₆ alkanes, branched C₁₀-C₁₆ esters, and mixtures thereof. Illustrative examples of a volatile high molecular weight hydrocarbon vehicle include isododecane, methylpentadecene, and isohexadecane. Specific examples of a volatile high molecular weight hydrocarbon vehicle include: isododecane sold as Permethyl 99A from Presperse (Somerset, N.J.); a combination of isododecane, hydrogenated tetradecenyl/methylpentadecene sold as SMART-5 from IMCD, The Netherlands; isohexadecane sold as Permethyl 101A from Presperse; the combination of C13-16 isoparaffin, C12-14 isoparaffin, and C13-15 alkane sold as SiClone SR-5 from Presperse; the combination of coconut alkanes and coco-caprylate/caprate sold as Vegelight 1214LC by Grant Industries (Elmwood Park, N.J.); coconut alkanes available from Biosynthis; and PPG-3 Benzyl ether ethylhexanoate sold as Crodamol SFX by Croda (Edison, N.J.).

In some embodiments a volatile high molecular weight hydrocarbon is used in conjunction with a second vehicle with a lower boiling point. A volatile high molecular weight hydrocarbon alone is typically not sufficiently volatile alone and leaves an oily feel on the skin due to this relatively low evaporation rate. Supplementing the volatile high molecular weight hydrocarbon with a second vehicle of lower boiling point will promote the pleasing feel in the skin by creating a desired overall compositional evaporation profile. A second vehicle is optionally an organic polyhalogenic vehicle or disiloxane. Disiloxane when used as a second vehicle is optionally of low viscosity such as 0.5 to 1.2 cSt. A second vehicle is optionally present at an amount that is 30% or less by weight, optionally 25% or less by weight.

In some embodiments, a volatile high molecular weight hydrocarbon is used in conjunction with an organic solvent of 5 carbons or fewer used at an overall amount of less than 5% by weight. An organic solvent used as such low levels was previously thought to be unable to promote solubility in a silicone based carrier of an active agent such as a vitamin A derivative (e.g. retinoid). This explains why the art recognized knowledge preferred higher levels of organic solvents such as ethanol. The high levels of ethanol (i.e. more than 15%, or to a lesser degree 5% or more) promotes skin irritation. It was unexpectedly discovered that a volatile high molecular weight hydrocarbon vehicle could be combined with the low level of organic solvent of 5 carbons or fewer to have multiple beneficial effects. The combination will fully solubilize otherwise not fully soluble active agents such as retinoids in organosiloxane carriers, will provide the appropriate overall evaporation profile of the overall material, and will protect against skin irritation. In some embodiments, an organic solvent of 6 carbons or fewer is ethanol, or isopropyl alcohol. An organic solvent of 5 carbons or fewer when used in conjunction with a volatile high molecular weight hydrocarbon vehicle is used at 5% or fewer by weight, optionally less than 5% by weight, optionally between 2-3 percent by weight. The organic solvent is preferably anhydrous.

A vehicle optionally has a boiling point less than 78° C. Optionally, a vehicle has a boiling point below 65° C. A vehicle is optionally present at a final concentration of about 2 percent to 40 percent w/w. Optionally, a vehicle is present at from 15 percent to 25 percent w/w. Optionally, a vehicle is present at 20% w/w. Optionally, a vehicle is present at from 1-5% w/w, optionally less than 5% w/w. It is appreciated that more than one vehicle is optionally present in an inventive composition. Optionally, 2, 3, 4, 5, 6, or more vehicles are present in an inventive composition. In some embodiments, a vehicle is not a perfluoroether.

The inventive composition is optionally formulated with levels of organic solvent. An organic solvent is optionally volatile at ambient temperatures and pressures. Optionally, less than 35% organic solvent is included. Optionally, less than 30% organic solvent is included. Optionally, the level of volatile organic solvent is less than 15 percent w/w. Optionally, an organic solvent is present at 5% or less w/w, optionally an organic solvent is present at less than 5% by weight. Optionally, an organic solvent is absent. An organic solvent is optionally an alcohol of 6 carbons or fewer. Optionally, an alcohol is an ethoxydiglycol, ethanol, or isopropyl alcohol. Optionally, an organic solvent is ethoxydiglycol present at 10 percent w/w or less. Optionally, ethoxydiglycol is present at 3 percent w/w. Organic solvent is optionally ethanol at less than 15% by weight, optionally less than 10% by weight, optionally 5% or less by weight, optionally less than 5% by weight. The level of organic solvent optionally does not induce noticeable drying or other toxic effects on the skin as opposed to systems that require volatile organic solvents such as ethanol at much higher concentrations. It is appreciated that more than one organic solvent is optionally present in an inventive composition. It is further appreciated that the inventive composition may be entirely free of organic hydrocarbon solvent of 6 or fewer carbons, optionally 5 or fewer carbons.

It is a particularly unexpected and surprising discovery of the subject invention that stable, fully solubilized solutions of active compounds in the carrier can be prepared with less than 15 percent w/w organic solvent when combined with a vehicle at 5 percent to 40 percent w/w. It is particularly surprising that a vehicle at 5 percent to 40 percent w/w can promote a stable fully solubilized solution with less than 5% organic hydrocabon solvent. The findings are particularly surprising when such an active agent is not fully soluble in either the carrier or the vehicle. It was unexpected that an active agent that is not fully soluble at a desired concentration in a volatile organosiloxane carrier and not fully soluble in a organic polyhalogenic vehicle, for example, would be fully soluble in a combination of volatile silicone carrier and organic polyhalogenic vehicle at that concentration, particularly when less than 5% of organic hydrocarbon solvent such as an alcohol (e.g. ethanol) is present, or such an organic solvent is absent. A composition is optionally free of other components to promote solubility of an active agent other than the volatile organosiloxane carrier and volatile vehicle. In some embodiments, the composition is free of other components to promote solubility of an active agent other than a volatile organosiloxane carrier that is a linear organopolysiloxane (optionally ethyl trisiloxane) and a volatile vehicle that is optionally perfluorohexane, pentafluoropropane, perfluorohexane, perfluorodecalin, methoxynonafluorobutane, disiloxane, a volatile high molecular weight hydrocarbon such as isododecane, ethanol or isopropyl alcohol at less than 15% by weight, PPG-3 Benzyl ether ethylhexanoate, or a combination thereof.

In many embodiments, the composition is substantially free of water, where the term substantially free of water is as low as practicably achievable in standard manufacturing conditions. The absence of water helps promote a uniform clear composition that is not subject to separation of oil and water phases and is not a suspension. Also, the absence of water will stabilize hydrophobic active agents in the compositions.

The composition is tailored to provide a clear solution of active agent that has a desirable overall evaporation profile and does not promote unacceptable skin irritation. The composition, therefore includes a volatile organosiloxane carrier that when combined with the volatile vehicle creates a clear solution in the absence of water. The composition has an evaporation rate of 0.01 to 15 mg/cm²/minute on contact with the skin or other surface at 30-32° C. in an air atmosphere at room temperature (25° C.) and standard atmospheric pressure (760 mmHg), and which is liquid at room temperature. Optionally, the composition has an evaporation rate ranging from 5-500 mg/cm²/minute at 25° C. and atmospheric pressure of 760 mmHg. In some embodiments, the composition has an evaporation rate ranging from 10 to 200 mg/cm²/minute at 25° C. and atmospheric pressure of 760 mmHg. Optionally, evaporation of 0.3-0.6 ml the volatile components when placed on the skin will be complete in 15-60 seconds, optionally 20-40 seconds, optionally 20-30 seconds. Such an evaporation profile of the composition will create the pleasing feel on the skin. Longer evaporation times produce an “oily” feel to the skin, whereas more rapid evaporation times leave the subject feeling as if nothing was applied to the skin possibly leading to overuse.

In addition, the composition does not promote unacceptable irritation of the skin. Skin irritation can be tested using a chromameter to measure skin redness produced by a test composition relative to that of a control or prior to application. An irritancy value in this standard art recognized test of 2.5 or less using a Minolta Chroma Meter is considered acceptable, but it is preferred that a composition have an irritancy value of less than 2, preferably less than 1.5, more preferably, 1.3 or less.

A composition of active agent, organosiloxane carrier, and volatile vehicle is a clear liquid representing complete or visually complete solubilization of the active agent in the volatile organosiloxane carrier in the presence of the vehicle. The presence of other additives may be used to alter the level of clarity, but optionally do not alter the solubilization level of the active agent. When a composition is a liquid, a clear solution is optionally has 90% transmittance at a desired wavelength relative to the same composition absent the active agent. Transmittance is optionally 91%, 92%, 93%, 94%, 95%, 96%, 97%, 98%, 99%, 99.5%, or in excess of 99.5% at a desired wavelength relative to the same composition absent the active agent. Methods of measuring transmittance using a spectrophotometer are well known in the art. A non-clear solution is opaque or possesses a haze or slight haze upon visual inspection.

Importantly, the compositions are stable in that the combination of the vehicle with the carrier will maintain the active agent in solution, and as such are capable of maintaining a clear solution at reduced temperature for a significant period of time. Illustratively, the composition will be free of active agent crystallization at a temperature of −20 to 10° C. for a period of 30 days or more. The compositions are in some embodiments free of active agent crystallization at a temperature of 4° C. for 30-90 days.

The composition optionally includes other additives or pharmaceutical carriers illustratively including: stabilizers such as the anti-oxidant BHT; surfactants illustratively Laureth-4; anti-oxidants illustratively vitamins C and E, and Green tea extract (i.e. Camellia sinensis) or SILOX GT from Collaborative Labs, Stony Brook, N.Y.; and emollients illustratively the mixture or single components of the emollient sold under the brand name SYMREPAIR available from Symrise, Teterboro, N.J. One of ordinary skill in the art readily appreciates additives suitable for use with the present invention such as to provide desired flow characteristics, absorption, evaporation, delivery of active agent, conversion of a prodrug, or other desired characteristic.

A composition optionally includes one or more pigments. Examples of pigments illustratively include inorganic or organic molecules such as molecules in the form of metal lakes. Pigments are illustratively made of titanium dioxide, zinc oxide, D&C Red No. 36 and D&C Orange No. 17, calcium lakes of D&C Red No. 7, 11, 31 and 34, barium lake of D&C Red No. 12, D&C Red No. 13 strontium lake, aluminum lakes of FD&C Yellow No. 5, of FD&C Yellow No. 6, of D&C Red No. 27, of D&C Red No. 21 and of FD&C Blue No. 1, iron oxides, manganese violet, chromium oxide and ultramarine blue.

The compositions of the invention are also optionally diluted to the appropriate active agent level for application by using other topically acceptable compounds or vehicles that are optionally miscible with a retinoid or other active agent of the invention. Other cosmetic additives are optionally employed either in the compositions of the invention or in those compositions when diluted with a suitable solvent.

Also provided are processes of fully solubilizing one or more retinoids in a solvent that is predominantly an oranosiloxane carrier. Fully solubilized active agents produce a clear material as defined herein. A process includes combining an active agent with a carrier at a concentration in which the active agent is not fully soluble in the volatile carrier. A process includes combining a volatile vehicle as defined herein with an active agent and a carrier to create a clear solution. An active agent is optionally vitamin A, or a vitamin A derivative such as a retinoid or derivative of a retinoid. Optionally, a vitamin A derivative is hydroxypinacolone retinoate or other esters or amides of 9- or 13-cis or trans-retinoids. It is known that vitamin A derivatives are poorly solubilized in volatile organosiloxane carriers. The combination of a volatile vehicle as defined herein surprisingly promotes visually complete solubilization of the vitamin A derivative in the carrier in the absence of water or greater than 5% organic solvent of 5 carbons or fewer such as ethanol.

Numerous skin or systemic conditions are treatable with the compositions illustratively including acne, wrinkles, dryness, eczema, psoriasis, actinic and nonactinic keratoses, rosaceous, among others. U.S. Pat. No. 3,932,665 describes retinal as a therapeutic agent in a method for treating acne by topical application. The topical administration of 5-fluorouracil for treatment of keratoses is described in U.S. Pat. No. 4,034,114. The inventive composition reduces the associated side effects that typically accompany topical or ophthalmologic administration of active agents while simultaneously providing a pleasing feel on the skin thereby improving patient compliance.

The inventive composition is suitable for topical delivery of an active agent. The inventive composition illustratively includes an active agent formulated in a carrier containing volatile organosiloxane and volatile vehicle. With such a carrier, active agent levels needed to achieve beneficial effects are minimized and the potential for irritant effects to the skin by the active agent (e.g. retinoids) are greatly diminished. Moreover, active agent is stable when formulated in the organosiloxane carrier compositions of the invention in contrast to other conventional carriers.

The compositions formulated as described herein with a retinoid as an active agent are optionally topically applied to the skin at a concentration that results in application of 0.005 to 1.0 weight percent retinoid, optionally 0.01 to 0.50 weight percent retinoid. A composition is optionally applied in the areas where fine lines, wrinkles, dry or inelastic skin or large pores are observed. Optionally, a moisturizer is applied with or after application of the inventive compositions to enhance the tactile comfort associated with application of the compositions and to enhance the wrinkle effacement and other benefits achieved by the compositions. An improved characteristic of the inventive composition is that the use of additional moisturizers is not required, but also not precluded.

Optionally, moisturizing efficacy is achieved in the compositions of the present invention containing the active agent, thereby precluding the need for a separate moisturizer. Therefore, optional compositions of the invention are formulated to include moisturizing components that are compatible with the volatile organosiloxane carrier to a level of up to 35% by weight of the final formulation. Preferred moisturizing ingredients are illustratively petrolatum, ethylhexyl palmitate, cholesterol fatty acid ceramide, and squalane. The addition of one or more moisturizing components is beneficial when the inventive composition is applied to previously dried skin or under conditions where dryness commonly occurs such as in cold climates, or winter months. Optionally, a moisturizing component is applied where the active agent itself has a drying effect such as when retinoid or 5-fluorouracil is applied.

With daily application of a retinoid containing composition, skin texture, color and tone will improve. Wrinkles and fine lines will be reduced with minimal irritant effects.

An inventive composition is optionally applied to the skin of a subject. A subject is optionally a patient. A subject is optionally a mammal such as humans, non-human primates, horses, goats, cows, sheep, pigs, dogs, cats, and rodents.

An inventive composition is optionally provided as a lotion, cream, gel, bar, ointment, or in pad form. Optionally, the composition is provided in a single use container the contents of which are applied directly to the stratum corneum of a subject or applied to an applicator pad for subsequent delivery to the subject.

A cooling effect is optionally observed upon application of the inventive composition. Cooling effect as used herein means reducing the temperature of skin, optionally, from about 1° C. to about 2° C. upon application. The cooling effect includes the effect that results from carrier or vehicle evaporation.

The inventive composition is optionally administered one to three times daily. Optionally, the inventive composition is delivered once daily. Optionally, the inventive composition is administered weekly, biweekly, monthly, or any subdivision thereof. It is appreciated that the inventive composition be administered for an amount of time suitable for efficacy of the active agent. Optionally, the inventive composition is administered for one to six weeks. Optionally, the inventive composition is administered indefinitely.

Also provided is a process of formulating an inventive composition optionally for pleasing administration to the skin of a subject. An inventive process illustratively includes making a first solution by solubilizing one or more active agents optionally in an organic solvent preferably performed with gentle mixing in low to no light conditions.

A second solution is made by mixing additives such as emollients and vitamins. The second solution is added to and mixed with the first solution. Mixing is preferably in the dark under gentle mixing conditions.

A third solution of carrier and vehicle is made and the third solution is added to the combined first and second solutions to form a composition. Mixing is optionally non-vortex, gentle mixing in low light or darkness. Mixing is preferably for 120 minutes. The composition is preferably stored under inert gas such as nitrogen gas.

It is appreciated that low to no light conditions are important should light sensitive components be present in the composition. In the absence of light sensitive components, the inventive process is optionally performed in ambient or other lighting conditions.

The inventive process is optionally performed at ambient temperature and pressure conditions. Optionally, the inventive process is performed by heating one or more components or solutions.

Various aspects of the present invention are illustrated by the following non-limiting examples. The examples are for illustrative purposes and are not a limitation on any practice of the present invention. It will be understood that variations and modifications can be made without departing from the spirit and scope of the invention. One of ordinary skill in the art readily knows how to synthesize or commercially obtain the reagents and components described herein.

### Example 1

A Formula A composition is mixed containing 3.0 percent ethoxydiglycol, 0.5 percent Laureth-4, 0.10 percent hydroxypinacolone retinoate, 0.05% BHT, 2.0 percent SYMREPAIR, 2.0 percent tetrahexyldecyl ascorbate, 0.50 percent tocopherol, 20 percent methoxynonafluorobutane, 1.0 percent SILOX GT, and the remainder Ethyl trisiloxane.

TABLE 1
Formula A

| | | Amount (% w/w | |
|---|---|---|---|
| | | unless otherwise | Commercial |
| Component | Tradename | noted) | Source |
| Phase 1 | | | |
| Ethoxydiglycol | Transcutol CG | 3 | Gattefosse |
| Laureth-4 | Brij 30 | 0.5 | Croda |
| Hydroxypinacolone retinoate | MDI-101 | 0.1 | Concert LLC |
| BHT | | 0.05 | |
| Phase 2 | | | |
| Hexyldecanol (and) Bisabolol | Symrepair | 2 | Symrise |
| (and) Cetyl hydroxyproline | | | |
| palmitate (and) Stearic acid | | | |
| (and) Brassica campestris | | | |
| (Rapeseed) sterols | | | |
| Tetrahexyldecyl ascorbate | BV-OSC | 2 | Barnet |
| Tocopherol USP | | 0.5 | |
| Phase 3 | | | |
| Ethyl trisiloxane | Silsoft ETS | qs 100% v/v | Momentiv |
| Methyl perfluorobutyl ether | CF-61 | 20 | 3M |
| (and) Methyl perfluoroisobutyl | | | Specialty |
| ether (methoxynonafluorobutane) | | | Materials |
| Cyclopentasiloxane (and) | Silox GT | 1 | BASF Beauty |
| Camellia sinensis leaf extract | | | Care |

Formula A is prepared by creating phase 1 containing Ethoxydiglycol (Transcutol CG purchased from Gattefosse, Toronto, ON, Canada), Laureth-4 (Croda, Edison, N.J.), Hydrocypinacolone retinoate (MDI-101, Concert LLC) and BHT by gentle mixing in a propeller mixer using low light conditions. Phase 2 is prepared separately. Phase 2 includes SYMREPAIR (Symrise, Inc., Teterboro, N.J.) which includes hexyldecanol, bisabolol, cetyl hydroxyproline palmitate, stearic acid, and Brassica campestris sterols. SYMREPAIR is mixed with tetrahexyldecyl ascorbate (BV-OSC, Barnet, Englewood Cliffs, N.J.) and tocopherol USP (DSM Nutritional Products, Inc.) in a propeller mixer until a clear solution fauns. Phase 1 is combined with phase 2 by slow addition with continuous, non-vortex propeller mixing protecting the solutions from light. Phase 3 is prepared by gentle propeller mixing at ambient temperature. Phase 3 includes ethyl trisiloxane (Silsoft ETS, Monentiv, Albany, N.Y.), methoxynonafluorobutane (3M Specialty Materials) and SILOX GT (combination of cyclopentasiloxane and Camellia sinesis leaf extract from BASF Beauty Care). The combined phases 1 and 2 are slowly added to phase 3 with continuous, non-vortex propeller mixing protected from the light. Mixing is continued for 120 minutes.

Formula A is transferred to opaque holding containers with nitrogen head-space for storage. 60 mL of Formula A is then transferred to 2 oz. amber glass bottles with a purified nitrogen gas head-space and stored protected from light until used.

### Example 2

A Formula B solution is prepared and stored as in Example 1 with the exception that Formula B includes perfluorohexane alone or in combination with perfluorodecalin and pentafluoropropane at 5% by weight in the place of methoxynonafluorobutane. Formula B also includes 8-12% disiloxane. An illustrative Formula B solution is illustrated in Table 2.

TABLE 2
Formula B

| | | Amount (% w/w | |
|---|---|---|---|
| | | unless otherwise | Commercial |
| Component | Tradename | noted) | Source |
| Phase 1 | | | |
| Ethoxydiglycol | Transcutol CG | 3 | Gattefosse |
| Laureth-4 | Brij 30 | 0.5 | Croda |
| Hydroxypinacolone retinoate | MDI-101 | 0.1 | Concert LLC |
| BHT | | 0.05 | |
| Phase 2 | | | |
| Hexyldecanol (and) Bisabolol | Symrepair | 2 | Symrise |
| (and) Cetyl hydroxyproline | | | |
| palmitate (and) Stearic acid | | | |
| (and) Brassica campestris | | | |
| (Rapeseed) sterols | | | |
| Tetrahexyldecyl ascorbate | BV-OSC | 2 | Barnet |
| Tocopherol USP | | 0.5 | |
| Phase 3 | | | |
| Ethyl trisiloxane | Silsoft ETS | qs 100% v/v | Momentiv |
| Perfluorohexane (and) | Fiflow BB61 | 5 | The |
| Perfluorodecalin (and) | | | Innovation |
| Pentafluoropropane | | | Company |
| Disiloxane (0.65 cSt) | Q7-9180 | 12 | Dow Corning |
| Cyclopentasiloxane (and) | Silox GT | 1 | BASF Beauty |
| Camellia sinensis leaf | | | Care |
| extract | | | |

### Example 3

A Formula C solution is prepared by combining three phases and stored as in Example 1. Formula C is as illustrated in Table 3.

TABLE 3
Formula C

| | | Amount (% w/w | |
|---|---|---|---|
| | | unless otherwise | Commercial |
| Component | Tradename | noted) | Source |
| Phase 1 | | | |
| Ethoxydiglycol | Transcutol CG | 3 | Gattefosse |
| Laureth-4 | Brij 30 | 0.5 | Croda |
| Hydroxypinacolone retinoate | MDI-101 | 0.1 | Concert LLC |
| BHT | | 0.05 | |
| Phase 2 | | | |
| Hexyldecanol (and) Bisabolol | Symrepair | 2 | Symrise |
| (and) Cetyl hydroxyproline | | | |
| palmitate (and) Stearic acid | | | |
| (and) Brassica campestris | | | |
| (Rapeseed) sterols | | | |
| Tetrahexyldecyl ascorbate | BV-OSC | 2 | Barnet |
| Tocopherol USP | | 0.5 | |
| Phase 3 | | | |
| Ethyl trisiloxane | Silsoft ETS | qs 100% v/v | Momentiv |
| Isododecane | Permethyl 99A | 10 | 3M |
| | | | Specialty |
| | | | Materials |
| Ethyl alcohol | | 5 | |
| Cyclopentasiloxane (and) | Silox GT | 1 | BASF Beauty |
| Camellia sinensis leaf | | | Care |
| extract | | | |

### Example 4

A Formula D solution is prepared by combining three phases and stored as in Example 1. Formula D is as illustrated in Table 4.

TABLE 4
Formula D

| | | Amount (% w/w | |
|---|---|---|---|
| | | unless otherwise | Commercial |
| Component | Tradename | noted) | Source |
| Phase 1 | | | |
| Ethoxydiglycol | Transcutol CG | 3 | Gattefosse |
| Laureth-4 | Brij 30 | 0.5 | Croda |
| Halobetasol | | 0.05 | Spectrum |
| Phase 2 | | | |
| Hexyldecanol (and) Bisabolol | Symrepair | 2 | Symrise |
| (and) Cetyl hydroxyproline | | | |
| palmitate (and) Stearic acid | | | |
| (and) Brassica campestris | | | |
| (Rapeseed) sterols | | | |
| Phase 3 | | | |
| Cyclotetrasiloxane | 244 Fluid | qs 100% v/v | Dow Corning |
| Perfluorohexane (and) | Fiflow BB61 | 5 | The |
| Perfluorodecalin (and) | | | Innovation |
| Pentafluoropropane | | | Company |
| Disiloxane (0.65 cSt) | Q7-9180 | 15 | Dow Corning |

### Example 5

A Formula E solution is prepared by combining three phases and stored as in Example 1. Formula E is as illustrated in Table 5.

TABLE 5
Formula E

| | | Amount (% w/w | |
|---|---|---|---|
| | | unless otherwise | Commercial |
| Component | Tradename | noted) | Source |
| Phase 1 | | | |
| Ethoxydiglycol | Transcutol CG | 3 | Gattefosse |
| Laureth-4 | Brij 30 | 0.5 | Croda |
| Hydroxypinacolone retinoate | MDI-101 | 0.1 | Concert LLC |
| BHT | | 0.05 | |
| Phase 2 | | | |
| Hexyldecanol (and) Bisabolol | Symrepair | 2 | Symrise |
| (and) Cetyl hydroxyproline | | | |
| palmitate (and) Stearic acid | | | |
| (and) Brassica campestris | | | |
| (Rapeseed) sterols | | | |
| Tetrahexyldecyl ascorbate | BV-OSC | 2 | Barnet |
| Tocopherol USP | | 0.5 | |
| Phase 3 | | | |
| Ethyl trisiloxane | Silsoft ETS | qs 100% v/v | Momentiv |
| PPG-3 Benzyl ether | Crodamol SFX | 10 | Croda |
| ethylhexanoate | | | |
| Disiloxane (0.65 cSt) | Q7-9180 | 25 | Dow Corning |
| Cyclopentasiloxane (and) | Silox GT | 1 | BASF Beauty |
| Camellia sinensis leaf | | | Care |
| extract | | | |

### Example 6

A Formula F solution is prepared by combining three phases and stored as in Example 1. Formula F is as illustrated in Table 6.

TABLE 6
Formula F

| | | Amount (% w/w | |
|---|---|---|---|
| | | unless otherwise | Commercial |
| Component | Tradename | noted) | Source |
| Phase 1 | | | |
| Ethoxydiglycol | Transcutol CG | 3 | Gattefosse |
| Laureth-4 | Brij 30 | 0.5 | Croda |
| Hydroxypinacolone retinoate | MDI-101 | 0.1 | Concert LLC |
| BHT | | 0.05 | |
| Phase 2 | | | |
| Hexyldecanol (and) Bisabolol | Symrepair | 2 | Symrise |
| (and) Cetyl hydroxyproline | | | |
| palmitate (and) Stearic acid | | | |
| (and) Brassica campestris | | | |
| (Rapeseed) sterols | | | |
| Tetrahexyldecyl ascorbate | BV-OSC | 2 | Barnet |
| Tocopherol USP | | 0.5 | |
| Phase 3 | | | |
| Ethyl trisiloxane | Silsoft ETS | qs 100% v/v | Momentiv |
| C13-C16 Isoparaffin (and) | SiClone SR-5 | 9 | Presperse |
| C12-C14 Isoparaffin (and) | | | |
| C13-C15 Alkane | | | |
| Disiloxane (0.65 cSt) | Q7-9180 | 28 | Dow Corning |
| Cyclopentasiloxane (and) | Silox GT | 1 | BASF Beauty |
| Camellia sinensis leaf | | | Care |
| extract | | | |

### Example 7

A comparator solution of Formula G is prepared by combining three phases and stored as in Example 1. Formula G is as illustrated in Table 7.

TABLE 7
Formula G

| | | Amount (% w/w | |
|---|---|---|---|
| | | unless otherwise | Commercial |
| Component | Tradename | noted) | Source |
| Phase 1 | | | |
| Ethoxydiglycol | Transcutol CG | 3 | Gattefosse |
| Laureth-4 | Brij 30 | 0.5 | Croda |
| Hydroxypinacolone retinoate | MDI-101 | 0.1 | Concert LLC |
| BHT | | 0.05 | |
| Phase 2 | | | |
| Hexyldecanol (and) Bisabolol | Symrepair | 2 | Symrise |
| (and) Cetyl hydroxyproline | | | |
| palmitate (and) Stearic acid | | | |
| (and) Brassica campestris | | | |
| (Rapeseed) sterols | | | |
| Tetrahexyldecyl ascorbate | BV-OSC | 2 | Barnet |
| Tocopherol USP | | 0.5 | |
| Phase 3 | | | |
| Cyclopentasiloxane | 245 Fluid | qs 100% v/v | Dow Corning |
| Ethyl Alcohol | | 50 | |
| Cyclopentasiloxane (and) | Silox GT | 1 | BASF Beauty |
| Camellia sinensis leaf | | | Care |
| extract | | | |

### Example 8

Formulas A-G are subjected to characterization for clarity and propensity to cause skin irritancy. Clarity is observed by visual observation by a single blinded evaluator and scored on a relative scale of C=clear, SH=slightly hazy, H=hazy, and O=opaque. Data are presented in Table 8.

The compositions of Formulas A-G are also analyzed for propensity to cause skin irritancy. Eight volunteer subjects in normal health and that do not exhibit any evidence of acute or chronic disease including dermatological or ophthalmologic problems are administered one of Formula A-H. The compositions are applied to both sides of the face in areas that are devoid of warts, nevi, moles, sunburn, suntan, scars and active dermal lesions. After an incubation time of 2 hours, measurements are made using a Minolta Chroma Meter as per the manufacturer's instructions. The values in the test area of each side of the face are averaged. Results are presented in Table 8.

TABLE 8

| | Formula | | | | | | |
|---|---|---|---|---|---|---|---|
| | A | B | C | D | E | F | G |
| Clarity | C | C | C | C | SH | C | C |
| Irritancy | 1.1 | 1.3 | 1.2 | 0 | 1.2 | 1.3 | 2.6 |

Each of the test formulations of Formulas A-F demonstrate low irritancy levels whereas the comparator solution (Formula G) that is typical of prior retinoid compositions produces significant irritation of the skin.

With respect to clarity, all of the formulas are clear with the exception of Formula E which shows a slight haze. This is due to the benzyl ether ethylhexanoate being less effective as a solvent at these levels. Increasing the benzyl ether ethylhexanoate level is expected to clarify the solution.

### Example 9

A solution of Formula H is prepared and stored as in Example 1. Formula H is as illustrated in Table 9.

TABLE 9
Formula H

| | | Amount (% w/w | |
|---|---|---|---|
| | | unless otherwise | |
| Component | Tradename | noted) | Commercial Source |
| Phase 1 | | | |
| Ethoxydiglycol | Transcutol CG | 3 | Gattefosse |
| Laureth-4 | Brij 30 | 0.5 | Croda |
| Hydroxypinacolone retinoate | MDI-101 | 0.1 | Concert LLC |
| BHT | | 0.05 | |
| Phase 2 | | | |
| Hexyldecanol (and) Bisabolol | Symrepair | 2 | Symrise |
| (and) Cetyl hydroxyproline | | | |
| palmitate (and) Stearic acid | | | |
| (and) Brassica campestris | | | |
| (Rapeseed) sterols | | | |
| Tetrahexyldecyl ascorbate | BV-OSC | 2 | Barnet |
| Tocopherol USP | | 0.5 | |
| Phase 3 | | | |
| Ethyl trisiloxane | Silsoft ETS | qs 100% v/v | Momentiv |
| Disiloxane (0.65 cSt) | Q7-9180 | 8.2 | Dow Corning |
| Ethyl alcohol | | 2.4 | |
| Cyclopentasiloxane (and) | SiloxGT | 1 | BASF Beauty Care |
| Camellia sinensis leaf | | | |
| extract | | | |

The composition of Formula H is tested for clarity as per Example 8. The blinded tester characterized the formulation as clear. Formula H also demonstrates low irritancy levels when measured in the skin irritancy test of Example 8 with an irritancy level of 1.1. This is due to the low irritancy of the disiloxane and the ethyl trisiloxane, as well as the very low level of ethanol which is less than in Formula C.

### Example 10

A second comparator solution of Formula I is made containing 46.3% Cyclomethicone-Tetramer; 35% Alcohol SD 40B Anhydrous; 5% Ethylhexyl Palmitate; 5% Octyl Dimethyl PABA; 2% Benzophenone-3; 2% Demineralized Water; 2% Neopentyl Glycol Dicaprate; 1.5% Ethyl Cellulose K5000; 0.22% Butylated Hydroxytoluene; and 1% Retinoid Blend. Formula B is prepared essentially as described in U.S. Pat. No. 4,826,828.

### Example 11

A split face test is performed by using test Formulas A-F and H or the comparator of Formulas G or I as follows. Twelve females aged 20 to 59 apply a single test formula to one side of their faces and a comparator to the other side once daily for eight weeks. Thin shavings of the skin on each side of the face are taken before the test begins and after the eight week test period. The skin shavings after the test are in better condition than those before the test in all twelve women in the test formula groups. The majority of the women in the comparator groups show skin improvement, but several do not. The skin of all women is both thicker and more organized after the test than before. All women in the test formula groups report improved moisture in the tested skin whereas the comparator group issues complaints of drying and cracking of the tested skin areas.

### Example 12

The ability of an electric current to flow through the stratum corneum provides an indirect measurement of the corneum's water content. The panelists who participated in the study in Example 11 are assessed for moisturization using an IBS impedance/conductance meter. At least twelve hours elapse between the last product application and the skin conductance measurement. The data demonstrate that the test formula treated sides are moister (higher conductance readings at all measurement time points) than comparator sides. The comparator side of the face fails to show similar levels of relative moisture content. Thus, the objective measurement and substantiation of the stratum corneum's electrical conductivity shows a significant enhancement in facial skin moisture content.

### Example 13

A test of the ability of the Formulas A-F and H relative to the comparators of Formulas G and I to reduce skin dryness is performed with or without supplemental moisturizer. Twelve panelists who demonstrate skin dryness upon repeated soap washing of the hands are selected to participate in this study. Initially, the panelists induce a condition of dryness by washing their hands with bar soap. The test formulations are applied daily to one hand while the other is left untreated to serve as a control side. Each hand is rated randomly by two trained evaluators who have no knowledge of which hand is treated. The evaluators use a stereomicroscope to assist them with their ratings. The results of this study are expected to demonstrate that the unmoisturized comparator hand shows additional dryness compared to the control hand. This level of dryness is improved by application of moisturizer after each comparator application. Formula A-F and H treated hands show marked improvement in moisture content. The addition of moisturizer after each Formula A-F and H application does not appreciably improve the treated skin moisture content. The Formula A-F and H benefits should persist for twenty-four hours after the final treatment indicating that the Formula A-F and H compositions provide effective long-lasting moisturization.

### Example 14

A Formula J solution is prepared wherein the active ingredient is benzoyl peroxide at 2.5 percent weight percent final. A phase 1 solution is prepared at ambient temperature by combining dimethyl isosorbide at 15% w/w final, ethanol (SD-Alcohol 40-B, 200 proof) at 4.7% w/w final, Laureth-4 at 1% w/w final, and benzoyl peroxide at 2.5% w/w final. The phase 1 ingredients are combined with continuous non-vortex propeller mixing.

Phase 2 is formed by combination of methyl perfluorobutyl ether (and) methyl perfluoroisobutyl ether (CF-61) at 35% w/w final and the remainder ethyl trisiloxane with continuous non-vortex propeller mixing until a clear solution is formed.

Phase 2 is slowly combined with phase 1 with continuous non-vortex propeller mixing. If a hazy solution is observed it will clarify upon standing for 24-48 hours at ambient temperature.

Formula J is stored in 60 ml volumes with absorbent applicator pads.

Formula K is prepared as in Formula J with the exception that Formula K includes 5% by weight perfluorohexane and 8% by weight disiloxane in the place of methoxynonafluorobutane.

### Example 15

Patients presenting with acne to a dermatologist provide informed consent to a split face test comparing Formula J or K of Example 14 with a commercially available benzoyl peroxide topical acne treatment of equal active ingredient concentration (STRIDEX POWER PADS, Blistex, Inc. Oak Brook, Ill.).

Fifteen females aged 20 to 39 apply Formula J or K to one side of their faces and the benzolyl peroxide comparator to the other side once daily for two weeks. Each subject is asked to record any side effects such as dryness, irritation, and perceived skin clarification. Both the Formula J and K, as well as the comparator are expected to demonstrate similar skin clarification. Subjects report less irritation and improved skin condition on the Formula J or K treated side relative to comparator.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. An active agent topical delivery composition comprising:
a volatile organosiloxane carrier;
an active agent selected from the group consisting of vitamin A or vitamin A derivatives, hydroxy acids, benzoyl peroxide, and combinations thereof; and
a volatile vehicle,
said composition having less than 15% organic hydrocarbon solvent of 6 carbon atoms or fewer.

2. The composition of claim 1 having an evaporation rate in air of 10 to 200 mg/cm²/minute at 25° C. and atmospheric pressure of 760 mmHg.

3. The composition of claim 1 with an irritancy value of 2 or less.

4. The composition of claim 1 that is a clear solution of said active agent.

5. The composition of claim 1 wherein said composition has less than 5% organic hydrocarbon solvent of 6 carbon atoms or fewer.

6. The composition of claim 1 wherein said agent is vitamin A or its derivatives wherein said vitamin A or its derivatives are present at between 0.001 to 2 weight percent.

7. The composition of claim 1 wherein said active agent is a vitamin A derivative selected from the group consisting of: retinal; retinoic acid; retinyl ester; retinol; tretinoin, isotretinoin, or esters thereof; an ester or amide of 13-trans retinoic acid; adapalene; tazarotene; or combinations thereof.

8. The composition of claim 1 wherein said hydroxy acid is salicylic acid, acetylsalicylic acid, or combinations thereof.

9. The composition of claim 1 wherein said agent is benzoyl peroxide present from about 1 to about 10 weight percent.

10. The composition of claim 1 wherein said organosiloxane carrier is a linear aliphatic polyorganosiloxane.

11. The composition of claim 7 wherein said organosiloxane carrier is ethyl trisiloxane.

12. The composition of claim 1 wherein said vehicle comprises a C₁-C₂₀ alkyl having a constituent of fluorine.

13. The composition of claim 1 wherein said vehicle comprises perfluorohexane, perfluorodecalin, disiloxane, isododecane, or combinations thereof.

14. The composition of claim 1 wherein said vehicle comprises a volatile high molecular weight hydrocarbon with 6 to 16 carbon atoms.

15. The composition of claim 14 wherein said vehicle further comprises an organic solvent of 5 carbons or fewer, or disiloxane.

16. The composition of claim 1 wherein said vehicle is present at from 5 percent to 40 percent by weight.

17. The composition of claim 1 wherein said vehicle is present from 10 to 25 percent by weight.

18. A process of treating a skin condition in a subject comprising:
administering to a subject the composition of claim 1.

19. The process of claim 18 wherein said composition contains less than 5% organic hydrocarbon solvent of 6 carbon atoms or fewer.

20. The process of claim 18 wherein said active agent is vitamin A or its derivatives present at between 0.001 to 2 weight percent.

21. The process of claim 18 wherein said volatile organosiloxane carrier is a linear aliphatic polyorganosiloxane.

22. An active agent topical delivery composition consisting essentially of:
an active agent selected from the group consisting of vitamin A or vitamin A derivatives, hydroxy acids, benzoyl peroxide, and combinations thereof;
a volatile organosiloxane containing carrier present as a predominant; and
a volatile vehicle, said volatile vehicle present at from 5 percent to 40 percent by weight;
the composition having less than 15% organic hydrocarbon solvent of 6 carbon atoms or fewer.

23. The composition of claim 22 wherein said organic hydrocarbon solvent is ethoxydiglycol.

24. The composition of claim 22 wherein said volatile vehicle is selected from the group consisting of perfluorohexane, perfluorodecalin, methoxynonafluorobutane, pentafluoropropane, disiloxane, a linear or branched C12-C16 alkane, a linear or branched C12-C16 alkane in combination with an organic hydrocarbon solvent of 5 carbons or fewer and at a concentration of less than 5% per weight, and combinations thereof.

25. The composition of claim 22 wherein said active agent is selected from the group consisting of: a retinal; retinoic acid; retinyl ester; retinol; tretinoin, isotretinoin, or esters thereof; an ester or amide of 13-trans retinoic acid; adapalene; tazarotene; and combinations thereof.

26. A process of solubilizing a derivative of vitamin A in a volatile organosiloxane carrier in the absence of 15% or greater organic hydrocarbon solvent of 5 carbon atoms or fewer comprising:
combining a vitamin A derivative with a volatile organosiloxane carrier and a volatile vehicle.

27. The process of claim 26 wherein said volatile organosiloxane carrier is present at 50% or greater.

28. The process of claim 26 wherein said step of combining includes said volatile vehicle at 5-40 percent by weight.

29. The process of claim 26 wherein said volatile vehicle is selected from the group consisting of perfluorohexane, perfluorodecalin, methoxynonafluorobutane, pentafluoropropane, disiloxane, isododecane, isododecane in combination with an organic hydrocarbon solvent of 5 carbons or fewer and at a concentration of less than 5% per weight, a linear or branched C12-C16 alkane other than isododecane, and combinations thereof.
